(19) United States
(12) Patent Application Publication
(10) Pub. No.: US 2008/0021198 A1
(43) Pub. Date: Jan. 24, 2008

(54) Modulators of protein phosphatase 2A and PP2A methyl esterase

(76) Inventors: Yigong Shi, Plainsboro, NJ (US); Yongna Xing, Plainsboro, NJ (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/548,883

(22) Filed: Oct. 12, 2006

Related U.S. Application Data
(60) Provisional application No. 60/725,724, filed on Oct. 12, 2005.

(51) Int. Cl. C07K 2/00; C07K 5/00; C07K 7/00; G01N 33/53

(52) U.S. Cl. 530/323; 435/7.1; 530/300; 530/328; 530/329; 530/330

(57) ABSTRACT

The disclosure relates to modulation of protein phosphorylation, including information derived from the structures and activities of the proteins designated protein phosphatase 2A (PP2A) and PP2A methyl esterase. The disclosure contained herein provides compounds and methods for identification of compounds that antagonize the function of PME, and thus reduce levels of PP2A demethylation activity. Over-expression or gain-of-function of PME contributes to a range of diseases such as cancer, thus inhibition of PME by antagonists may provide a strategy for therapeutic intervention.

### CROSS-REFERENCE TO RELATED APPLICATIONS

This application claims the benefit of U.S. Provisional Patent Application No. 60/725,724, filed on Oct. 12, 2005, the contents of which are incorporated herein by reference.

### STATEMENT REGARDING FEDERALLY SPONSORED RESEARCH

Not Applicable

### THE NAMES OF THE PARTIES TO A JOINT RESEARCH AGREEMENT

Not Applicable

### INCORPORATION-BY-REFERENCE OF MATERIAL ON DISC

Not Applicable

### BACKGROUND

The disclosure contained herein generally relates to the field of modulation of protein phosphorylation. In particular, the disclosure relates to the structure of protein phosphatase 2A methyl esterase (PME), which may be used as the basis for screening and rational design of inhibitors of PME. These inhibitors may be useful for restoring the function of protein phosphatase 2A and for inhibiting tumor growth.

Reversible protein phosphorylation, namely protein phosphorylation and dephosphorylation, is a fundamental regulatory mechanism in all aspects of biology (Hunter T (1995) Cell 80:225-236). Reversible protein phosphorylation was first discovered by Edmond Fischer and Edwin Krebs in 1955, when they purified glycogen phosphorylase and showed that the enzyme could be converted from an inactive to an active form through transfer of a phosphate group from ATP to the protein (Krebs E & Fischer E (1955) J Biol Chem 216:113-120; Krebs E & Fischer E (1955) J Biol Chem 216:121-132). Since the, hundreds of protein kinases and phosphatases have been identified in the human genome (Alonso A et al. (2004) Cell 117:699-711; Manning G et al. (2002) Science 298:1912-1934), each specific for a different group of proteins and/or distinct biological pathway.

The human genome contains 107 tyrosin protein phosphatases (Alonso A et al. (2004) supra), but only a few Serine-Threonine (Ser/Thr) protein phosphatases. The Ser/Thr protein phosphatases are classified into three structurally distinct families: PPM, PPP and FCP/SCP. Protein phosphatase 2A (PP2A) belongs to the PPP family and is a major Ser/Thr phosphatase that may be involved in many essential aspects of cellular function and regulation (Janssens V & Goris J (2001) Biochem J 353:417-439; Virshup D (2000) Curr Opin Cell Biol 12:180-185; Lechward K et al. (2001) Acta Biochim Pol 48:921-933). By some estimates, PP2A may account for the majority of all Ser/Thr phosphatase activities in mammalian cells. PP2A plays a principal role in cell cycle regulation, cell growth control, development, regulation of multiple signal transduction pathways, cytoskeleton dynamics and cell mobility. The essential function of PP2A is reflected by the fact that the catalytic subunit of PP2A is among the most conserved enzymes across species (Cohen P et al. (1990) FEBS Lett 268:355-359).

The multi-task functions of PP2A reside in its complex composition and regulation. Unlike other phosphatases, PP2A contains three subunits (FIG. 1). The core component of PP2A consists of a 36-kDa catalytic subunit, known as the C subunit, and a 65-kDa scaffold protein, known as the A or PR65 subunit. In mammalian cells, both the A and C subunits have two isoforms, alpha (α) and beta (β), which share very high sequence similarity (Hemmings B et al. (1990) Biochemistry 29:3166-3173; Stone S et al. (1987) Biochemistry 26:7215-7220; Green D et al. (1987) Proc Natl Acad Sci USA 84:4880-4884; Arino J et al. (1988) Proc Natl Acad Sci USA 85:4252-4256). All isoforms are ubiquitously expressed in different tissues and cell types, although Aα and Cα appear to be more abundant than Aβ and Cβ (Khew-Goodall Y & Hemmings B (1988) FEBS Lett 238:265-268; Hendrix P et al. (1993) J Biol Chem 268:7330-7337). To gain full activity toward specific substrates, the PP2A core component, the A-C hetero-dimer, interacts with a third regulatory subunit, known as the B subunit, to form a hetero-trimeric holoenzyme. The B subunit has 4 subfamilies in humans: PR55/B, PR61/B′, PR72/B″, and PR93/PR110/B′″, with at least four members in PR55/B, five members in PR61B′, five members in PR72/B″ and two members in PR93/PR110/B′″ (Janssens V & Goris J (2001) supra; Lechward K et al. (2001) supra). Unlike the A and C subunits, the sequent similarity among the B subunits is very low and the expression levels of various B subunits are highly diverse depending upon cell types and tissues. In this regard, the B subunits may determine the substrate specificity as well as the spatial and temporal functions of PP2A.

The large number of PP2A configurations and the broad substrate specificities may explain why there are only a few Ser/Thr protein phosphatases in the human genome and further, may explain why PP2A is the dominant Ser/Thr phosphatase. The many configurations of PP2A are also consistent with the principal roles of PP2A in multiple cellular functions and the fact that deregulation PP2A function is related to many different diseases, ranging from neural degenerative disorders (Tsujio I et al. (2005) FEBS Lett 579:363-372; Rametti A et al. (2004) J Biol Chem 279:54518-54528; Sun L et al. (2003) Neuroscience 118:1175-1182; Planel E et al. (2001) J Biol Chem 276:34298-34306; Goedert M et al. (2000) J Neurochem 75:2155-2162) to various types of cancers (Janssens V et al. (2005) Curr Opin Genet Dev 15:34-41). Tumorigenic transformation of cells by viral proteins, such as small T antigen (ST), is also mediated through interference with PP2A function (Moreno C et al. (2004) Cancer Res 64:6978-6988; Hahn W et al. (2002) Mol Cell Biol 22:2111-2123; Yang C et al. (2005) Mol Cell Biol 25:1298-1308). Viral proteins may replace the cellular B subunit and take over the host cell machinery, leading to uncontrolled cell growth and division. In this regard, PP2A represents an attractive target for potential therapeutic intervention in a range of diseases.

The alpha4 protein was initially identified as a component of receptor signaling complexes in mammalian lymphocytes and was later found to be broadly expressed (Inui S et el. (1995) J Immunol 154:2714-2723; Chuang E et al. (2000) Immunity 13:313-322; Everett A & Brautigan D (2002) Dev Dyn 224:461-464). Alpha4 interacts with the C subunit of PP2A and this binding appears to displace the C subunit from the A and B subunits (Murata K et al. (1997) Proc Natl Acad Sci USA 94:10624-10629; Chen J et al. (1998) Biochem Biophys Res Commun. 247:827-832; Zolnierowicz S (2000) Biochem Pharmacol 60:1225-1235) (FIG 1). This observation is consistent with a mutagenesis study in which the C subunit of PP2A was found to use a partially overlapping surface for binding to the alpha4 protein and to the A subunit (Prickett I & Brautigan D (2004) J Biol Chem 279:38912-38920). Remarkably, binding by alpha4 appears to alter the substrate specificity of PP2A. Alpha 4 inhibits apoptosis and is essential for cell survival (Kong M et al. (2004) Science 306:695-698). Alpha4 also bridges the interaction between the C subunit of PP2A and MIDI, a ubiquitin ligase that targets the PP2A C subunit for degradation (Schweiger S & Schneider R (2003) Bioessays 25:356-366). Together, these observations suggest that elevated levels of alpha4 may counteract the function of PP2A. Since the alpha4 protein is essential for cell survival, targeted ablation of this protein in cancer cells might be a potential strategy for therapeutic intervention.

The function of PP2A is highly regulated, not just through association with different regulatory subunits, but also through two major forms of post-transactional modification, phosphorylation and methylation. There are six highly conserved residues “TPDY₃₀₇FL₃₀₉” at the carboxyl terminus of the PP2A C subunit. Phosphorylation of reside Tyr307 is thought to inactivate the enzyme (Chen J et al. (1992) Science 257:1261-1264). Experimental evidence indicates that reversible methylation of the carboxy-terminal residue Leu309 of the PP2A C subunit provides another important mechanism for the regulation of PP2A activity (Lee J & Stock J (1993) J Biol Chem 268:19192-19195; Lee J et al. (1996) Proc Natl Acad Sci USA 93:6043-6047; Xie H & Clarke S (1993) J Biol Chem 268:13364-13371; Xie H & Clarke S (1994) Biochem Biophys Res Commun 203:1710-1715; Xie H & Clarke S (1994) J Biol Chem 269:1981-1984). The trimeric holoenzyme was mainly purified in a methylated form (Moreno C et al. (2000) J Biol Chem 275:5257-5263; De Baere I et al. (1999) Biochemistry 38:16539-16547), whereas the A-C hetero-dimer was fully demethylated (De Baere I et al. (1999) supra; Bryant J et al. (1999) Biochem J 339:241-246). Methylation of Leu309 in the C subunit regulates the recruitment of the regulatory B subunits (Wei H et al. (2001) J Bio Chem 276:1570-1577; Yu X et al. (2001) Mol Biol Cell 12:185-199; Wu J et al. (2000) Embo J 19:5672-5681; Tolstykh T et al. (2000) Embo J 19:5682-5691). Interestingly, methylation of the C subunit occurs throughout the cycle, but the level of methylation is temporarily decreased at the G₀/G₁ and G₁/S transitions (Turowski P et al. (1995) J Cell Biol 129:397-410).

Two specific enzymes have been identified which regulate methylation of the PP2A C subunit (FIG. 1). Addition of a methyl group to Leu309 of the C subunit of PP2A is catalyzed by a unique protein phosphatase methyltransferase (PMT) (Lee J & Stock J (1993) supra; De Baere I et al. (1999) supra). PMT contains a conserved S-adenosyl methionine (SAM)-binding motif, but is distinct from other protein methyltransferases. Removal of the methyl group is catalyzed by a specific methylesterase, protein phosphatase methylesterase (PME) (Lee J et al. (1996) supra). PME exhibits very limited sequence similarity to other esterases, and consequently the catalytic triad residues of PME are not fully identified in the prior art. The substrate for both PMT and PME is thought to be the core A-C hetero-dimer, but not the free C subunit or the hetero-trimeric holoenzyme. Despite the importance of these two enzymes, no structural information is available for any mammalian PMT or PME.

A delicate balance between protein kinases and protein phosphatases is crucial for cells to properly function and divide. Many kinases have been demonstrated to function as oncogenes or proto-oncogenes (Hananhan D & Weinberg R (2000) Cell 100:57-70; Broach J & Levine A (1997) Curr Opin Genet Dev 7:1-6), and consequently the phosphorylation of cellular targets is generally associated with a positive signal for cell growth and proliferation. Compared to the well-documented oncogenic kinase signaling events, considerably less is known about how and when protein phosphatases can terminate a signal. In the prior art, only a very limited number of Ser/Thr phosphatases have been identified which antagonize the signaling events. An increasing body of observations has led to the conclusion that PP2A is an important tumor suppressor protein and a principle guardian against tumorigenic transformation. Because of the diverse functions of PP2A and its involvement in multiple pathways that regulate cell growth and proliferation, deregulation of PP2A is associated with a large profile of cancers.

The first line of evidence suggesting that PP2A may be involved in cancer and cancer progression is the discovery that PP2A is the cellular target of okadaic acid (Bialojan C & Takai A (1988) Biochem J 256:283-290). Because okadaic acid is a potent tumor inducer, its specific inhibition of PP2A function led to the proposal that PP2A is a negative regulator of cellular growth and proliferation. The second line of evidence is that both the α and the β isoforms of the A subunit have been identified as tumor suppressors. Mutations in the A subunit that result in compromised binding to the B or C subunits of PP2A, or a total absence or substantial reduction of the A subunit, has been reported to be closely associated with a variety of primary human tumors (Wang S et al. (1998) Science 282:284-297; Takagi Y et al. (2000) Gut 47:268-271; Calm G et al. (2000) Oncogene 19:1191-1195; Ruediger R et al. (2001) Oncogene 20:1892-1899; Colella S et al. (2001) Int J Cancer 93:798-804; Suzuki K & Takahashi K (2003) Int J Oncol 23:1263-1268). Furthermore, an N-terminally truncated form of the B subunit PR61/B′γ1 was found to be associated with a higher metastatic state of melanoma cells (Ito A et al. (2000) Embo J 19:562-571; Ito A et al. (2003) Am J Pathol 162:81-91; Koma Y et al. (2004) Histol Histopathol 19:391-400). PR61/B′γ1 specifically targets PP2A to focal adhesions for the dephosphorylation of paxillin. The N-terminally truncated form of PR61/B′γ1 resulted in a PP2A hetero-trimer that failed to perform this function, leading to increased migration and metastasis.

More importantly, PP2A was found to antagonize the function of an important oncogene c-Myc, a central regulator of the cell cycle and of cell growth (Yeh E et al. (2004) Nat Cell Bio 6:308-318; Sears R et al. (2000) Genes Dev 14:2501-2514; Watnick R et al. (2003) Cancer Cell 3:219-231). Deregulated expression of c-Myc occurs in a broad range of human cancers and is often associated with aggressive, poorly differentiated phenotypes. A complex signal transduction network has ben deciphered that controls the stabilization, accumulation, and degradation of c-Myc. Upon phosphorylation at reside Thr58, the phosphate group at Ser62 can then be removed by PP2A, and c-Myc is then targeted to the ubiquitin-proteasome pathway for degradation (Yeh E et al. (2004) supra), The SV40 small T antigen (SI) appears to transform cells by blocking the function of PP2A. ST competes with a regulatory B subunit, possibly PR61/B′γ1, to associate with the PP2A A-C hetero-dimer and thus blocks its normal function, leading to c-Myc stabilization (Moreno C et al. (2004) supra).

The present lack of structural information for the aforementioned PP2A isoforms and associated subunits prevents their use as targets for drug screening and rational drug design. Thus, a need exists to identify the structural features and regulatory mechanisms of these proteins that underlie their ability to facilitate phosphorylation and dephosphorylation of their specific substrates. Additionally, there is no structural information available for the two enzymes which regulate methylation of PP2A: PME and PMT. Thus, a need exists to identify structural features of these two enzymes that may allow elucidation of their regulatory mechanisms, and thus regulation of PP2A activity.

### SUMMARY

Embodiments of the disclosure address the need for structural information on PME which may be useful in the identification of inhibitors of protein phosphatase 2A methyl esterase (PME). In an embodiment, a compound is disclosed which comprises a mimetic of a peptide capable of binding to a binding pocket of a PME, wherein the mimetic may have a three dimensional structure complementary to the binding pocket of the PME defined by atomic coordinates of a substrate-PME complex. In another embodiment, the mimetic may have a three dimensional structure which is represented by a model that deviates from the atomic coordinates of the c-terminus of the substrate by a root mean square deviation of less than 10 angstroms. The c-terminus of the substrate may be represented by a peptide which comprises amino acids involved in hydrogen bonding and van der Waals interactions with residues in the binding pocket of PME. In an embodiment, the substrate may be PP2A or a carboxyl-terminal portion of PP2A, and a residue within the binding pocket may be the active Ser156 of PME.

In a further embodiment, the c-terminus of the substrate may be represented by a computationally modeled peptide which comprises amino acids from the c-terminus of protein phosphatase 2A (PP2A).

In an embodiment, the mimetic may be a peptide. In a further embodiment, the peptide may have at least one amino acid replaced with a modified amino acid, and/or at least one bond replaced with a peptide bond substitute.

An embodiment of the disclosure is a peptide capable of binding to a binding pocket of a protein phosphatase 2A methyl esterase (PME). An additional embodiment is a peptide capable of binding to a catalytic site of PME which may further be capable of inhibiting PME catalytic activity.

An embodiment is a peptide of sequence Arg-Arg-Thr-Pro-Asp-Tyr-Phe-Z, where Z may be Leucine, Leucinal or LPMM, and wherein the peptide may also be capable of inhibiting PME catalytic activity.

The disclosure also provides an embodiment which is a synthetic peptide comprising the sequence selected from the group consisting of X₁X₂H₃X₄X₅X₆X₇X₈ (SEQ ID No. 8); X₂X₃X₄X₅X₆X₇X₈ (SEQ ID No. 9); X₃X₄X₅X₆X₇X₈ (SEQ ID No. 10) X₄X₅X₆X₇X₈ (SEQ ID No. 11); and X₅X₆X₇X₈ (SEQ ID No. 12); wherein X₁ is Arg, Lys or Gln or a mimetic of Arg, Lys or Gln; X₂ is Arg, Lys or Gln or a mimetic of Arg, Lys or Gln; X₃ is Thr, Ser or Val or a mimetic of Thr, Ser or Val; X₄ is Pro or Ala or a mimetic of Pro or Ala; X₅ is Asp or Glu or a mimetic of Asp or Glu; X₆ is Tyr or Phe or a mimetic of Tyr or Phe; X₇ is Phe or Tyr or a mimetic of Phe or Tyr; and X₈ is Leu, Ile, Val, Leucinal or LPMM or a mimetic of Leu, Ile, Val, Leucinal or LPMM.

A further embodiment provides for synthetic peptides of sequence Arg-Arg-Thr-Pro-Asp-Tyr-Phe-Leu (SEQ ID No. 2), Thr-Pro-Asp-Tyr-Phe-Leu (SEQ ID No. 7) and Asp-Tyr-Phe-Leu (SEQ ID No. 6). In an embodiment, the peptides may have at least one amino acid replaced with a modified amino acid, and/or at least one bond replaced with a peptide bond substitute. The peptide may also be able to inhibit the catalytic activity of PME.

An embodiment of the disclosure provides a method of identifying a compound that inhibits catalytic activity of protein phosphatase 2A methyl esterase (PME), comprising obtaining a set of atomic coordinates defining a three dimensional structure of a crystal of a substrate-PME complex that effectively diffracts X-rays for the determination of atomic coordinates to a resolution of 5 Angstroms or better, selecting a compound that mimics the substrate binding to the catalytic site on the PME by performing structure based drug design with the atomic coordinates obtained in step (a), wherein said selecting is performed in conjunction with computer modeling, contacting the compound with the PME, and detecting binding of the compound with the catalytic site of the PME, wherein the compound is selected if it is capable of inhibiting PME catalytic activity. In an embodiment, the substrate may be PP2A or a carboxyl-terminal portion of PP2A.

In an embodiment, the selecting performed in conjunction with computer modeling may be selecting a mimetic which is represented by a model that deviates from the atomic coordinates of the substrate by a root mean square deviation of less than 10 angstroms, wherein the substrate is represented by a peptide which comprises amino acids involved in hydrogen bonding and van der Waals interactions with the catalytic site of PME. In a further embodiment, performing structure based drug design may comprise computational screening of one or more databases of chemical compound structures to identify candidate compounds which have structures that are predicted to interact with the catalytic site of the PME.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

Aspects, features, benefits and advantages of the embodiments herein will be apparent with regard to the following description, appended claims, and accompanying drawings, where:

FIG. 1 is a schematic diagram illustrating the regulation of PP2A. The core component of PP2A is the A-C heterodimer. Assembly of the A-C heterodimer with a regulatory subunit B forms the PP2A holoenzyme. Reversible methylation of the C-terminal leucine residue of the C subunit, by PMT and PME, regulates the interaction between the A-C heterodimer and the B subunits. The C subunit can also form a complex with the α4 protein.

FIG. 2 illustrates structural models for PME as determined by solution of the atomic coordinates from a crystal of PME. A ribbon diagram is shown on the left and a space filling model on the right. The putative catalytic residue SER156 is labeled in both views.

FIG. 3 illustrates a bar graph comparing the activity of WT and mutant forms of PME. The assayed material is visualized on a sodium dodecyl sulfate polyacrylamide gel electrophoresis (SDS-page) by coomasie staining, which is shown in the inset at bottom.

FIG. 4 illustrates the chemical structures of some PME inhibitors.

FIG. 5 illustrates a chromatogram for the purification of full-length PME by gel filtration. Relevant fractions from the gel filtration column are visualized by coomasie staining of an SDS-page, which is shown in the inset at bottom.

FIG. 6A illustrates identification of the trypsin-resistant PME core by trypsin digestion with increasing amounts of trypsin, as visualized on a coomasie stained SDS-page.

FIG. 6B illustrates chromatograms from the fractionation of trypsin-digested PME by anion exchange chromatography. The peak in each chromatogram represents an isolated stable PME core, and is visualized by coomasie stained SDS-page in each inset.

FIG. 7A is a schematic diagram of an in vitro assay for PME activity.

FIG. 7B is a plot comparing the esterase activity of full-length PME and the PME core.

FIG. 8 illustrates a purification profile for the PP2A A-C heterodimer from bovine brain by anion exchange chromatography. Relevant fractions from the anion exchange column are visualized on a coomasie stained SDS-page, and are shown in the inset at bottom.

FIG. 9 illustrates a scheme for synthesis of leucinal.

FIG. 10 illustrates a scheme for synthesis of LPMM.

### DETAILED DESCRIPTION

Before the present compositions and methods are described, it is to be understood that they are not limited to the particular compositions, methodologies or protocols described, as these may vary. It is also to be understood that the terminology used in the description is for the purpose of describing the particular versions or embodiments only, and is not intended to limit their scope in the present disclosure which will be limited only by the appended claims. Various scientific articles, patents and other publications are referred to throughout the specification. Each of these publications is incorporated by reference herein in its entirety.

It must also be noted that as used herein and in the appended claims, the singular forms “a”, “an”, and “the” include the plural reference unless the context clearly dictates otherwise. Thus, for example, reference to an “antagonist” is a reference to one or more antagonists and equivalents thereof known to those skilled in the art, and so forth. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

“Optional” or “optionally” means that the subsequently described event or circumstance may or may not occur, and that the description includes instances where the event occurs and instances where it does not. In addition, the word “comprising” as used herein means “including, but not limited to”. Throughout the specification of the application, various terms are used such as “primary”, “secondary”, “first”, “second”, and the like. These terms are words of convenience in order to distinguish between different elements, and such terms are not intended to be limiting as to how the different elements may be utilized.

As used herein, “isolated” means altered or removed from the natural state through human intervention. For example, a PME naturally present in a living animal is not “isolated,” but a synthetic PME, or a PME partially or completely separated from the coexisting materials of its natural state is “isolated.” An isolated PME can exist in substantially purified form, or can exist in a non-native environment such as, for example, a cell into which the PP2A has been delivered.

The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics, as further described below.

By “pharmaceutically acceptable”, it is meant the carrier, diluent or excipient must be compatible with the other ingredients of the formulation and not deleterious to the recipient thereof. As used herein, the term “pharmaceutically acceptable salts, esters, amides, and prodrugs” refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention.

The terms “therapeutically effective” or “effective”, as used herein, may be used interchangeably and refer to an amount of a therapeutic composition of embodiments of the present invention (e.g., one or more of the peptides or mimetics thereof). For example, a therapeutically effective amount of a composition comprising a mimetic is a predetermined amount calculated to achieve the desired effect. As used herein, an “effective amount” of the antagonist or mimetic is an amount sufficient to cause antagonist mediated inhibition of PME, and thus modulate PME and PP2A activity in a range of diseases, such as cancer. As used herein, the term “cancer” refers to any type of cancer, including, but not limited to, ovarian cancer, leukemia, lung cancer, colon cancer, CNS cancer, melanoma, renal cancer, prostate cancer, breast cancer, and the like.

Reversible protein phosphorylation, namely protein phosphorylation and dephosphorylation, is a fundamental regulatory mechanism in all aspects of biology. Protein phosphatase 2A (PP2A) is a dominant Ser/Thr protein phosphatase in mammalian cells and a principal tumor suppressor protein against oncogenic transformation. The core component of PP2A consists of the scaffolding subunit (A subunit) and the catalytic subunit (C subunit). Methylation of the C subunit, controlled by the PP2A methyl transferase (PMT) and the PP2A methyl esterase (PME) is essential to the function of PP2A. Further, the alpha4 protein antagonizes the normal function of PP2A by forming a complex with the C subunit of PP2A.

Despite the importance of PP2A function in mammalian cells, numerous fundamental questions remain unanswered. In particular, there is a lock of structural information on PP2A, its binding proteins, and modifying enzymes. In the prior art, the only piece of direct structural information on mammalian PP2A and its regulatory enzymes is the crystal structure of the A subunit of human PP2A (Groves M et al. (1999) Cell 96:99-110). Without accurate structural information, it is difficult to gain a deeper understanding of the regulation of PP2A, or the interaction of the core enzyme with regulatory subunits or with other cellular or viral proteins, or the mechanisms of modification enzymes such as PMT and PME. To address these fundamental issues, we have carried out systematic X-ray crystallographic and biochemical analyses of the PP2A core component, its regulatory proteins, and its modifying enzymes.

We have determined the crystal structure of PME to 2.1 Angstrom resolution using multi-wavelength anomalous dispersion (MAD). This structural information may provide insight into the enzyme function and substrate binding, and may further provide a strategy for inhibition of PME. Such information facilitates the design and screening of specific inhibitors of PME, which may be useful in clinical applications for anti-cancer therapy. Thus, an embodiment of the current disclosure is peptides, peptidomimetics or mimetics thereof, or small molecule compounds which may act as antagonists of PME demethylation activity. Further, these peptides, peptidomimetics or mimetics thereof, or small molecule compounds may be useful in the treatment of tumor growth, cancer or cancer progression.

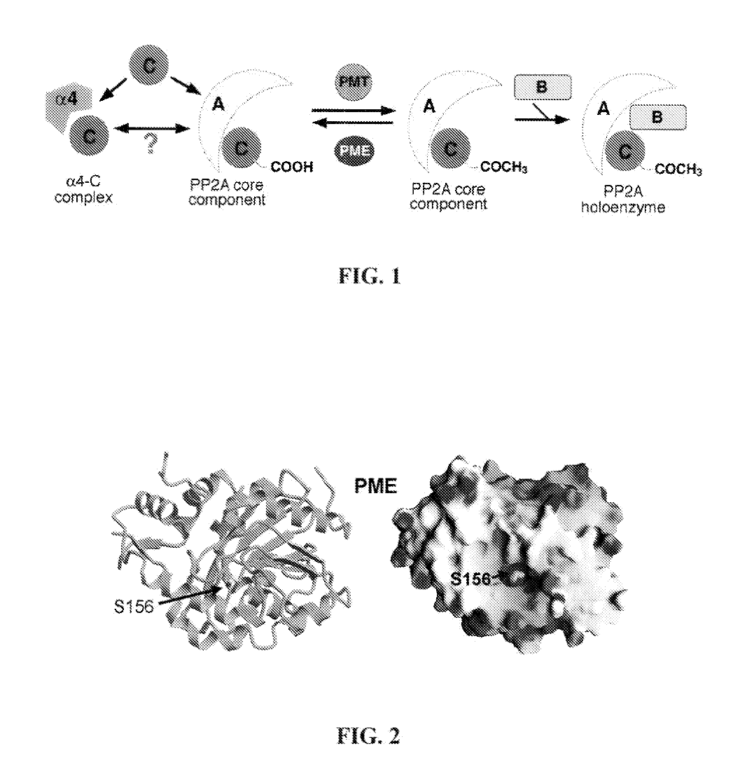

The structural features of PME (FIG. 2) show a prominent pocket leading to Ser156, the putative catalytic residue. This pocket may likely be the binding site for the C-terminus of the catalytic subunit of PP2A. As such, this region may provide a useful target location for inhibitors of PME activity. Enzymatic removal of the methyl group from the methylated leucine reside Leu309 of the PP2A subunit may involve a nucleophilic attack on the carbonyl carbon atom of Leu309 by the activated oxygen atom in the catalytic serine (Ser156). This may result in an intermediate with a tetrahedral configuration. Compounds that mimic the structure of the PP2A carboxyl terminal peptide with an appropriate tetrahedral configuration may have the ability to inhibit the enzymatic function of PME.

Thus an embodiment of the disclosure provides a compound comprising a mimetic of a peptide capable of binding to a binding pocket of a protein phosphatase 2A methyl esterase (PME), wherein the mimetic may have a three dimensional structure complementary to the binding pocket of the PME defined by atomic coordinates of PME or a substrate-PME complex. In an embodiment, the mimetic may have a three dimensional structure which is represented by a model that deviates from the atomic coordinates of the c-terminus of the substrate by a root means square deviation of less than 10 angstroms. The c-terminus of the substrate may be represented by a peptide which comprises amino acids involved in hydrogen bonding and van der Waals interactions with the residues in the binding pocket of PME, which may include the active Ser156. In additional embodiment, the c-terminus of the substrate may be represented by a computationally modeled peptide which comprises amino acids from the c-terminus of protein phosphatase 2A (PP2A).

In another embodiment, the compound may be identified by a method which includes obtaining a set of atomic coordinates defining a three dimensional structure of a crystal of a substrate-PME complex that effectively diffracts X-rays for the determination of atomic coordinates to a resolution of 5 Angstroms or better. A compound may then be selected that mimics the substrate binding to a catalytic site on the PME by performing structure based drug design with the atomic coordinates. Selecting a mimetic may be performed in conjunction with computer modeling and includes selecting a mimetic which is represented by a model that deviates from the atomic coordinates of the substrate by a root mean square deviation of less than 10 angstroms, wherein the substrate may be represented by a peptide which comprises amino acids involved in hydrogen bonding and van der Waals interactions with the catalytic site of PME. In one embodiment, the mimetic may be a peptide, wherein at least one amino acid may be replaced with a modified amino acid, and at least one bond may be replaced with a peptide bond substitute.

Another embodiment of the disclosure provides a compound which is a mimetic of a peptide capable of binding to a binding pocket on PME. The compound may be identified by a method which includes obtaining a set of atomic coordinates defining a three dimensional structure of a crystal of PME that effectively diffracts X-rays for the determination of atomic coordinates to a resolution of 5 Angstroms or better. A compound may then be selected by structure based drug design using the atomic coordinates for the active site of PME, and may be performed in conjunction with computer modeling. The structure based drug design may be directed compound design or random compound design. In one embodiment, the mimetic may be a peptide, wherein at least one amino acid may be replaced with a modified amino acid, and at least one bond may be replaced with a peptide bond substitute.

These compounds, which are peptides or mimetics thereof, may then be contacted with the PME and binding may be detected. This binding may be done in a cell-free assay, or may be done in a cell-culture assay. The compound may be considered a mimetic if binding of the compound with the catalytic site of the PME modulates PME catalytic activity. The compound may also be considered a mimetic if binding of the compound with a binding pocket on PME modulates PME catalytic activity.

In a further embodiment, the substrate may be defined by a model of the carboxyl-terminal residues of PP2A. The model may be defined by the structure of the substrate in the co-crystalline model of a substrate-PME complex, wherein the substrate is preferably PP2A or a peptide mimic thereof, or by a computational model of the carboxyl-terminal residues of PP2A. The computational model may be derived from structural homology modeling or by molecular dynamics simulations of PP2A or a peptide representing the carboxyl-terminal residues of PP2A.

In yet a further embodiment, the mimetic may be selected using computational screening of one or more databases of chemical compound structures to identify candidate compounds which have structures that are predicted to interact with the catalytic site of the PME. In yet another embodiment, the mimetic may be capable of inhibiting the activity of PME.

Another embodiment of the present invention is a method of treating a patient with an inhibitor of a phosphatase methyl esterase comprising administering a compound that has a three-dimensional structure complimentary to the binding pocket of the PME defined by the atomic coordinates of a substrate-PME complex.

In yet another embodiment of the instant disclosure, these small molecules or peptidomimetics or mimetics which are antagonists of PME activity may be used as a therapeutic for the treatment of diseases, including but not limited to cancer. Accordingly, an embodiment of the disclosure comprises administering to a cell a therapeutically effective amount of the compounds to inhibit the activity of PME, wherein inhibiting the activity may be useful for the treatment of cancer. In another embodiment, the cell is contained within a tissue, and the tissue preferably is located in a living organism, preferably an animal, more preferably a mammal, and most preferably a human.

These later embodiments of the disclosure are carried out by formulating the mimetics described herein as pharmaceutical preparations or therapeutic compositions for administration in a subject. Such a pharmaceutical preparation constitutes another aspect of the disclosure. For example, in some aspects, the disclosure is directed to a pharmaceutical composition comprising a compound, as defined above, and a pharmaceutically acceptable carrier or diluent, or an effective amount of a pharmaceutical composition comprising a compound as defined above.

The compounds of the present invention can be administered in the conventional manner by any route where they are active. Administration can be systemic, topical, or oral. For example, administration can be, but is not limited to, parenteral, subcutaneous, intravenous, intramuscular, intrapertioneal, transdermal, oral, buccal, or ocular routes, or intravaginally, by inhalation, by depot injections, or by implants. Thus, modes of administration for the compunds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion subcutaneously over a period of about 15 minutes to about 24 hours. Formulations of injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coating for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism.

Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention disclosure may also be administered in combination with other active ingredients, such as, for example, adjuvants, protease inhibitors, or other compatible drugs or compounds where such combination is seen to be desirable or advantageous in achieving the desired effects of the methods described herein.

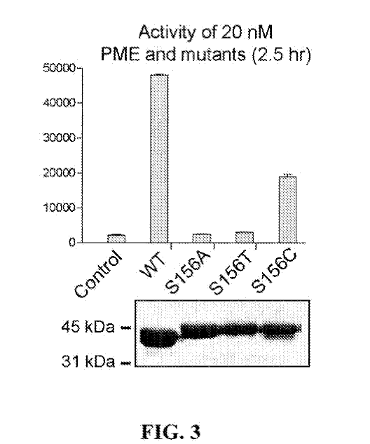

The crystal structure of protein methyl esterases of the present invention and a biochemical analysis as disclosed herein may provide information about the enzyme's active site. Based on the structural features, we may identify the active site residues using mutagenesis and as in vitro assays. Ser156 in PME appears to correspond to the catalytic serine in the structure of the sialyl acetyl methylesterase from plant (Forouhar F et al. (2005) Proc Natl Acad Sci USA 102:1773-1778). We investigated whether Ser156 may also be the catalytic residue in PME by generating three missense mutations, S156A, S156T, and S156C. As shown in FIG. 3, mutation of Ser156 to alanine completely abolished the enzyme activity, while mutation to threonine and cysteine retained 10% and 40% of the enzyme activity, respectively. These results may provide support for Ser156 as a conserved catalytic reside for enzyme function.

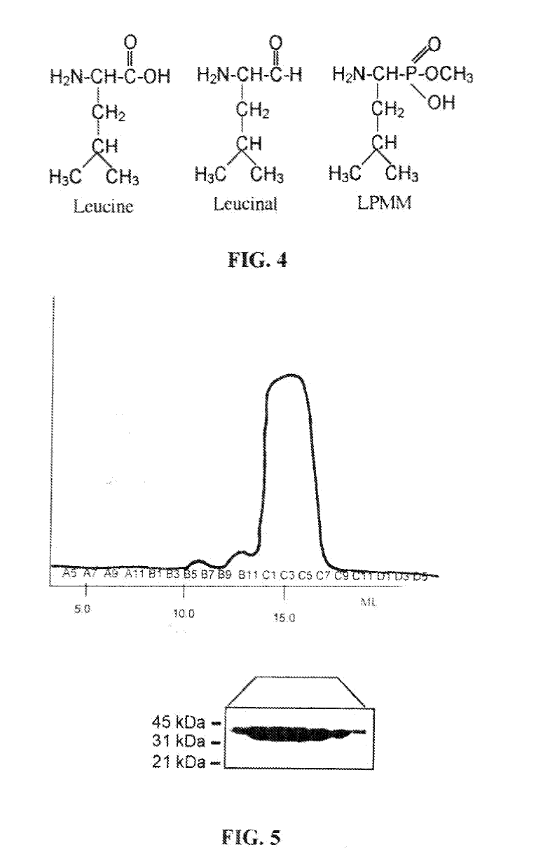

Two major categories of compounds which may act as inhibitors of PME are provided herein. The first category includes Leucinal (FIG. 4), N-acetyl Leucinal and the peptide derivative Arg-Arg-Thr-Pro-Asp-Tyr-Phe-Leucinal (SEQ ID NO. 1), in which, Leucinal replaces the leucine reside of the synthetic peptide derived from the carboxy-terminal eight residues of the PP2A C subunit Arg-Arg-Thr-Pro-Asp-Tyr-Phe-Leu (SEQ ID NO. 2). The α-aminoaldehyde of the Leucinal is a much stronger electrophile than other aldehyde compounds (Andersson L et al. (1982) Biochemistry 21:4177-4180; Andersson L & Wolfenden R (1982) Anal Biochem 124:150-157). All of these compounds may form a covalent linkage with the catalytic residue Ser156, which resembles the tetrahedral intermediate.

The second category of compounds is phosphite derivatives, including the phosphonic acid analogue of leucine monomethyl ester (abbreviated LPMM; FIG. 4), N-acetyl LPMM and its peptide derivative, Arg-Arg-Thr-Pro-Asp-Tyr-Phe-LPMM (SEQ ID NO. 3). This category of compounds may mimic the tetrahedral intermediates of the enzyme reaction without covalent attachment to the active Ser156 (Lejczak B et al (1989) Biochemistry 28: 3549-3885). Further, these peptide derivatives may resemble the substrates, and hence have affinity to the enzyme.

Thus, an embodiment of the disclosure provides for a peptide or peptidomimetic, or mimetic of the carboxy-terminal residues of the PP2A C subunit. The peptide may be of the sequence SEQ ID Nos. 1-3, or may be a mimetic of SEQ ID Nos. 1-3. The peptide or peptidomimetic, or mimetic of the carboxy-terminal residues of the PP2A C subunit may contain more than eight amino acids, such as, for example, nine, ten, eleven or twelve amino acids in length. Thus, a peptide of an embodiment with nine or ten amino acids may be Thr-Arg-Arg-Thr-Pro-Asp-Tyr-Phe-Leu (SEQ ID NO. 4) or Val-Thr-Arg-Arg-Thr-Pro-Asp-Tyr-Phe-Leu (SEQ ID NO. 5), respectively. The peptide or peptidomimetic, or mimetic of the carboxy-terminal residues of the PP2A C subunit may contain less than eight amino acids, such as, for example, four or six amino acids in length. Thus, a peptide of an embodiment with four or six amino acids may be Asp-Tyr-Phe-Leu (SEQ ID NO. 6) or Thr-Pro-Asp-Tyr-Phe-Leu (SEQ ID NO. 7).

A further embodiment of the disclosure provides for a peptide, or mimetic thereof, of the sequence:

| | |
|---|---|
| X₁X₂X₃X₄X₅X₆X₇X₈, | (SEQ ID NO. 8) |
| X₂X₃X₄X₅X₆X₇X₈, | (SEQ ID NO. 9) |
| X₃X₄X₅X₆X₇X₈, | (SEQ ID NO. 10) |
| X₄X₅X₆X₇X₈, | (SEQ ID NO. 11) |
| or | |
| X₅X₆X₇X₈, | (SEQ ID NO. 12) |

where X₁ may Arg, Lys or Gln or a mimetic of Arg, Lys or Gln, X₂ may Arg, Lys or Gln or a mimetic of Arg, Lys or Gln, X₃ may be Thr, Ser or Val or a mimetic of Thr, Ser or Val, X₄ may Pro or Ala or a mimetic of Pro or Ala, X₅ may Asp or Glu or a mimetic of Asp or Glu. X₆ may Tyr of Phe or a mimetic of Tyr of Phe, X₇ may Phe or Tyr or a mimetic of Phe or Tyr, and X₈ may Leu, Ile, Val, Leucinal or LPMM or a mimetic of Leu, Ile, Val, Leucinal or LPMM. Further, a compound of an embodiment may be Leucine, Leucinal, or LPMM or a mimetic of Leucine, Leucinal, or LPMM.

Further, another embodiment is directed to a therapeutic composition comprising a peptide of sequence SEQ ID Nos. 1-7, or a mimetic of SEQ ID Nos. 1-7. An additional embodiment is directed to a therapeutic composition comprising a peptide of sequence SEQ ID Nos. 8-12, or mimetic of SEQ ID Nos. 8-12. A further embodiment is directed to a therapeutic composition comprising a compound which may be may be Leucine, Leucinal, or LPMM or a mimetic of Leucine, Leucinal, or LPMM.

A method of treating a tumor growth in a patient with cancer comprising administering a therapeutically effective amount of a peptide of sequence SEQ ID Nos. 1-7, or a mimetic of SEQ ID Nos. 1-7. An additional embodiment is directed to a method of treating a tumor growth in a patient with cancer comprising administering a therapeutically effective amount of sequence SEQ ID Nos. 8-12, or a mimetic of SEQ ID Nos. 8-12. A further embodiment is directed to a method of treating a tumor growth in a patient with cancer comprising administering a therapeutically effective amount of a compound which may be may by Leucine, Leucinal, or LPMM or a mimetic of Leucine, Leucinal, or LPMM.

We can characterize the inhibition of PME by these various substrate analogs and inhibitory compounds, peptides, peptidomimetics and mimetics thereof, including but not limited to Leucine, Leucinal, LPMM, and their derivatives. Inhibition of PME may represent a positive regulation of the enzymatic activity of PP2A. Using the in vitro enzymatic assay disclosed herein, we may test the PME enzymatic activity in the presence of different concentrations of the substrate analogs and inhibitory compounds, peptides, peptidomimetics and mimetics thereof.

PMT and PME perform two opposing functions, methylation and demethylation, respectively, on the same substrate, the C subunit of PP2A. In addition, PMT and PME also directly interact with the PP2A A-C hetero-dimer. Thus, PMT and PME may exhibit some common features in substrate recognition, catalysis, and binding to PP2A. Although PME and PMT do not share extensive sequence similarity, comparison of PME and PMT primary sequences revealed short stretches of amino acid peptides with reasonable sequence homology. These regions may correspond to shared substrate-binding or PP2A-binding motifs in the structures.

We have determined the crystal structure of PMT to 2.2 Angstrom resolution. This structural information provides insight into the enzyme function and substrate binding, and may further provide a strategy for activation of PMT. Such information facilitates the design and screening of specific activators of PMT, which may be useful in clinical applications for anti-cancer therapy. As such, an additional embodiment of this disclosure is peptides. peptidomimetics or mimetics thereof, or small molecule compounds which may act as agonists of PMT methylation activity. Further, these peptides, peptidomimetics or mimetics thereof, or small molecule compounds may be useful in the treatment of tumor growth, cancer or cancer progression.

We have carried out biochemical characterization of PME and regulation of the PP2A core enzyme by PMT, PME, and alpha4 using highly purified recombinant PME, PMT, alpha4, PP2A core component, and the A-C hetero-dimer proteins. We have developed an in vitro system to study the enzymatic function of PMT and PME and to investigate the biochemical mechanisms by which alpha4, PME, and PMT regulate the function of PP2A.

PP2A is an important tumor suppressor protein and a principal guardian against tumorigenic transformation. The activity of PP2A is down-regulated in most, if not all, types of cancer. Elucidating the function and mechanisms of PP2A may be important to understanding the tumorigenic pathways. In addition, multiple layers of regulation make PP2A a very attractive target for the potential therapeutic intervention of cancer. For example, selective inhibition of PME, selective reduction in the expression level of alpha4 protein, and disruption of interaction between alpha4 and the PP2A C subunit may augment the normal cellular function of PP2A, which in turn may inhibit tumor growth. Taken together, these experimental results may provide a method for screening and rational design of modulators of PP2A activity. For example, antagonists of PME activity may lead to enhanced PP2A activity and the identification of a therapeutic compound useful for tumor suppression and cancer treatment. Alternately, agonists of PMT activity may also lead to enhanced PP2A activity and the identification of a therapeutic compound useful for tumor suppression and cancer treatment. In this respect, the information in this disclosure may reveal insights into the regulation of PP2A that may facilitate clinical applications.

This disclosure further provides a method for screening for small molecule compounds, peptides, peptidomimetics and mimetics that may act as agonists of PMT or antagonists of PME. These compounds, peptides, peptidomimetics or mimetics thereof may act to enhance the activity of PP2A and may thus be useful in clinical applications as anti-cancer therapies.

In another embodiment of the instant disclosure, the PME binding peptides which are antagonists or the PMT binding peptides which are agonists are modified to produce peptide mimetics by replacement of one or more naturally occurring side chains of the 20 genetically encoded amino acids (or D amino acids) with other side chains. For example, the other side chains may contain groups such as alkyl, lower alkyl, cyclic 4-, 5-, 6-, to 7-membered alkyl, amide, amide lower alkyl, amide di(lower alkyl), lower alkoxy, hydroxyl, carboxy and the lower ester derivatives thereof, and with 4-, 5-, 6-, to 7-membered heterocyclics. For example, praline analogs can be made in which the ring size of the proline reside is changed from 5 members to 4, 6 or 7 members. Cyclic groups can be saturated or unsaturated, ad is unsaturated, can be aromatic or non-aromatic. Heterocyclic groups can contain one or more nitrogen, oxygen, and/or sulfur heteroatoms. Examples of such groups include furazanyl, furyl, imidazolindyl, imidazolyl, imidazolinyl, isothiazolyl, isozolyl, morpholinyl (e.g. morpholino), oxazolyl, piperazinyl (e.g. 1-piperazinyl), piperidyl (e.g. 1-piperidyl, piperdino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl, (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidometics may also have amino acid residues that have been chemically modified by phosphorylation, sulfonation, biotinylation, or the addition or removal of other moieties.

Based upon the information provided herein, a variety of techniques are available for constructing peptide mimetics with the same or similar desired biological activity as the corresponding native but the more favorable activity then the peptide with respect to solubility, stability, and/or susceptibility to hydrolysis or proteolysis (Morgan et al. (1989) Ann Rep Med Chem 24:243-252). Certain preferred peptidomimetic compounds are based upon the amino acid sequence of the peptides of the disclosure. Often, peptidomimetic compounds are synthetic compounds having a three dimensional structure (i.e., a “peptide” motif) based upon the three dimensional structure of a selected peptide. The peptide motif provides the peptidomimetic compound with the desired biological activity, i.e. binding to PME or PMT, wherein the binding activity of the mimetic compound is not substantially reduced, and is often the same as or greater than the activity of the native peptide on which the mimetic was modeled. Peptidomimetic compounds can have additional characteristics that enhance their therapeutic application, such as increased cell permeability, greater affinity and/or avidity and prolonged biological half-life.

Based upon the information and data provided in the present application, peptidomimetic design strategies are available in the art (Ripka et al. (1988) Curr Opin Chem Biol 2:441-452; Hruby et al. (1997) Curr Opin Chem Biol 1:114-119; Hruby et al. (2000) Curr Med Chem 9:945-970). One class of peptidomimetic applicable to the present invention provides for mimicing a backbone that is partially or completely non-peptide, but mimics the peptide backbone atom-for-atom and comprises side groups that likewise mimic the functionality of the side groups of the native amino acid residues. Several types of chemical bonds e.g. ester, thioester, thioamide, retroamide, reduced carboyl, dimethylene and ketomethylene bonds, are known in the art to be generally useful substitues for peptide bonds in the construction or protease resistant peptidomimetics. Another class of peptidomimetics comprises a small non-peptide molecule that binds to another peptide or protein, but which is not necessarily a structural mimetic of the native peptide.

Yet another class of peptidomimetics has arisen from combinatorial chemistry and the generation of massive chemical libraries. These generally comprise novel templates which, though structurally unrelated to the native peptide, posses necessary functional groups positioned on a non-peptide scaffold to serve as “topographical” mimetics of the original peptide (Ripka A et al. (1998) supra).

This invention and embodiments illustrating the method and materials used may be further understood by reference to the following non-limiting examples.

### EXAMPLES

### Example 1

### Determination of the Crystal Structure of PME and its Complex with Substrate or Inhibitor

PME is an essential enzyme that regulates the activity of PP2A. PME exhibits a very low level of sequence homology to other known esterases. Neither the mechanism of this enzyme nor its interaction with the PP2A core component is understood.

Expression and Purification of the Full-Length PME

The full-length PME protein contains 386 amino acids, with a poly-glutamate region similar to that found in the alpha4 protein. Using standard recombinant DNA technology, the full-length PME may be cloned into a T7-based expression plasmid (pET30a, Novagen) with an N-terminal His6 tag followed by a TEV protease cleavage site. The expression level of the recombinant PME protein was high in bacterial host BL21(DE3) either for overnight induction with 0.5 mM IPTG at room temperature, or incubation for 3-5 hours at 37° C.

The soluble fraction of the His6-tagged PME in the E. coli lysate may be purified over a Ni-NTA agarose column, and further purified by anion-exchange chromatography (Source-15Q, Pharmacia). The N-terminal His6 tag may be removed by overnight incubation of the protein with TEV protease at 4° C. with a 1:20 molar ratio of protease to protein with simultaneous dialysis against cell lysate buffer to remove imidazole. After removal of the uncleaved protein by Ni-NTA agarose column, the resulting protein may be purified by anion-exchange chromatography (Source-15Q, Pharmacia) and gel-filtration chromatography (Superdex-200, Pharmacia). The full-length PME of the disclosure using these techniques may be purified to greater than 98% homogencity (FIG. 5). The concentration of PME may be determined by spectroscopic measurement at 280 nm. A final yield of approximately 8 mg of pure PME protein per liter of bacterial culture may be expected.

Limited Proteolysis of PME and Characterization of the Resulting PME Core

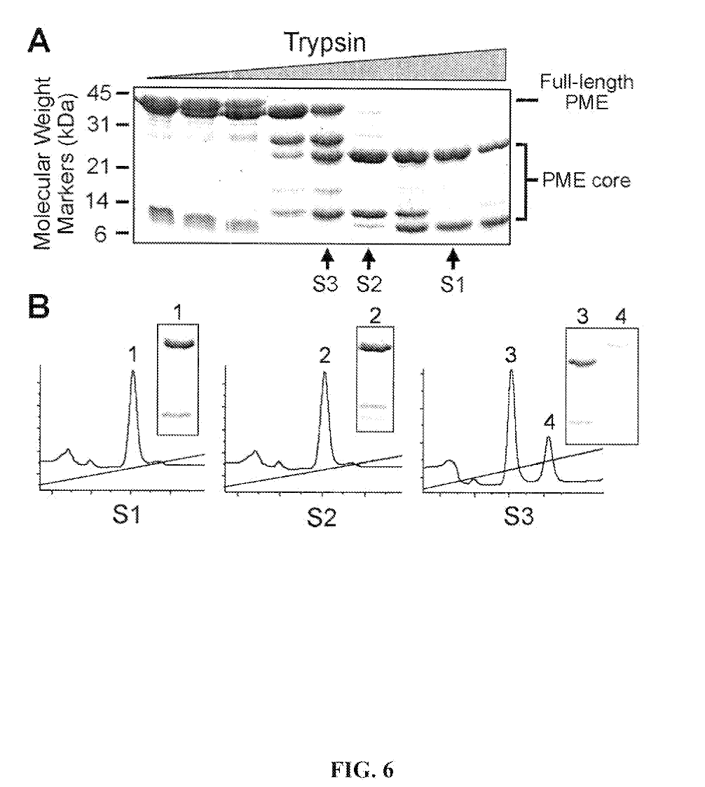

We performed systematic crystallization screens for the full-length PME, using both sparse matrix kits and systematic grids screens from a number of commercial companies. Unfortunately, a large proportion, up to 35%, of all screened conditions yielded phase separation. One possibility for this observation is that the full-length PME may contain flexible loops on the surface that impede crystallization. In this case, an effective strategy to generate structural core domains of a protein may be to apply limited proteolysis so that the flexible surface loops may be removed without affecting the functional core. We used a number of proteases, including trypsin, subtilisin, and chemotrypsin, to digest the full-length PME. Trypsin gave the most promising result (FIG. 6A). Samples using three different concentrations of trypsin were individually fractionated on an analytical anion exchange column to determine the optimal concentration of enzyme that would give the best digestion result (FIG. 6B). Trypsin digestion produced two fragments, at 26 and 8 kDa, that co-migrated on the anion exchange column and hence may be readily separated from other fragments (FIG. 6B). Analysis by N-terminal peptide sequencing and mass spectroscopy indicated that the N-terminal fragment (residues 1-38) and the internal poly-glutamate motif (residues 249-272) had been removed by limited proteolysis. This experiment identified a trypsin-resistant structural core of PME, which represents an attractive target for crystallization.

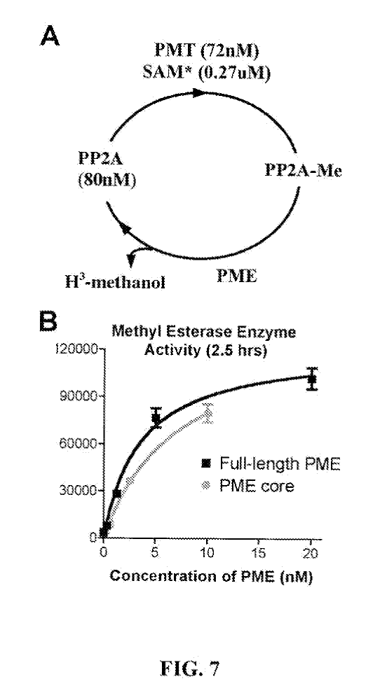

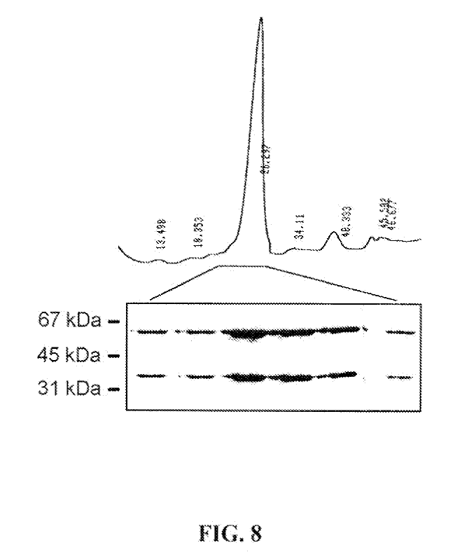

To characterize the trypsin-resistant structural core of PME, we compared its methyl esterase activity to that of the full-length PME. Radiolabeled H³-SAM is mixed with 7.2 μM PP2A A-C dimer and 0.2 μM PMT to generate methylated A-C dimmer (FIG. 7A). The substrate, PP2A A-C hetero-dimer, may be purified from bovine brain. Release of H³-methanol may be initiated by addition of full-length PME enzyme or the PME core. Concentration dependent PME enzyme activity may be determined by the release of H³-methanol after 2.5 hours or overnight incubation of the reaction mixture at 37° C. The results indicate that the PME core exhibits a similar methyl esterase activity to that of the full-length PME (FIG. 7B).

Crystallization and Data Collection of PME Core

Crystals of PME core may be grown at 4° C. by the hanging-drop vapor-diffusion method by mixing PME core (15 mg/ml) with an equal volume or reservoir solution containing 24-26% Jeffamine-2001 (v/v), 200 mM NaCl, and 5 mM DTT. The crystals appeared after 1-2 days and reached a maximum size within one week. The crystals are in the primitive space group P3121, with unit cell parameters a=b=82.5 Angstroms, c=90.8 Angstroms, alpha=beta=90°, gamma=120°, and contain one protein molecule in each asymmetric unit.

Diffraction data were collected using an ADSC quantum 315 CCD detector at the X25 beamline of National Synchrotron Light Source (NSLS) at Brookhaven National Laboratory (BNL). The crystals appear as thin needles and diffract X-rays beyond 2.0 Angstrom resolution. To prevent potential crystal decay problems, we equilibrated crystal of the PME core in a cryoprotectant buffer containing 34% Jeffamine-2001 (v/v), 200 mM NaCl, and 5 mM DTT, and flash-froze the crystals in liquid nitrogen and kept the crystals frozen under nitrogen stream at −170° C. during data collection. A complete 2.05 Angstrom native data set and a complete 2.6 Angstrom seleno-methionine MAD dataset were collected (Table 1).

Structure Determination and Preliminary Refinement

The MAD data may be processed using the HKL suite of programs. The selenium atom sites may be located by SOLVE (Terwilliger T & Berendzen J (1999) Acta Crystallogr D55:849-861). Initial MAD phases calculated with the program MLPHARE (Collaborative Computational Project N—The CCP4 suite: programs for protein crystallography (1994) Acta Crystallogr D50:760-763) had a mean figure of merit of 0.62 to 2.6 Angstrom (Table 1). The phases were modified and extended to 2.1 Angstrom resolution using CNS (Brunger A et al. (1998) Acta Crystallogr. D54:905-921). The experimental electron density map contained contiguous electron density for most of the backbone atoms in PME as well as the majority of the side chains. An atomic model was built into the electron density map using the program O (Jones T et al. (1991) Acta Crystallogr A47:110-119). Refinement was performed using CNS.

Crystallization of PME Bound to its Substrates, Substrate Analogs and Inhibitors.

To understand how PME recognizes its substrate and to determined the catalytic mechanism of PME, we may crystallize PME bound to substrates, substrate analogs, and/or inhibitors. Substrates that may be used are SEQ ID Nos. 1-4, Leucinal, or LPMM. Methylated versions of the peptides with SEQ ID. Nos. 1-4 may also act as substrates. The crystals may be formed from pre-formed complexes pf the PME-substrate, or may be formed by soaking the substrate into a crystal of the PME. For the unmethylated peptide, co-crystallization may be carried out with the wild type (WT) PME. For the methylated peptide, co-crystallization may be carried out with the mutant form of PME—mutation at the catalytic residue Ser156 to Ala or Thr. This may be an efficient way to prevent substrate hydrolysis during crystallization.

Determination of the structure of PME bound to substrate peptides or mimetics thereof, or to an intact substrate may be carried out by molecular replacement using the structure of the PME core. Comparison of the structure of PME alone and PME bound to these compounds may reveal insights into the mechanism and function of PME.

TABLE 1
Crystallographic Analysis of the PME Core

| Data Sets | Native | Peak | Inflection | Remote |
|---|---|---|---|---|
| Wavelength (Angstrom) | 1.10 | 0.9793 | 0.9795 | 0.9500 |
| Resolution (Angstrom) | 99-2.05 | 99-2.6 | 99-2.6 | 99-2.6 |
| Unique reflections | 25,077 | 11,458 | 11,469 | 11,460 |
| Completeness | 99.5% | 99.3% | 99.3% | 99.3% |
| (outershell) | (97.3%) | (99.9%) | (99.9%) | (100%) |
| R_{sym} (outer shell) | 0.0074 (0.29) | 0.125 (0.33) | 0.123 (0.41) | 0.122 (0.36) |
| Data redundancy | 2.6 | 7.6 | 7.6 | 7.6 |
| Average [symbol] /(sigma)(outer shell) | 26.4(2.8) | 19.5(2.8) | 17.8(2.6) | 16.6(2.2) |
| Anomalous difference (%) | | 11.9 | 10.0 | 10.6 |
| Cullis R factor | | 0.82 | 0.85 | 0.90 |
| Phasing power | | 1.03 | 0.91 | 0.68 |
| Mean FIG. of Merit (20-2.6 Angstroms) | | 0.62 | | |

### Example 2

### Determination of the Crystal Structure PMT and its Complex with Cofactor SAM or Enzymatic Reaction Product SAH

PMT uses SAM as a cofactor and catalyzes the transfer of the methyl group from SAM to the carboxyl terminal leucine residue of PP2A C subunit. While PMT does contain a conserved SAM binding motif, the rest of the sequences are different from other methyl transferases. To understand the mechanism of PMT and how PMT regulates PP2A, we have determined the crystal structure of human PMT.

Expression and Purification of the Human PMT Protein

The full-length PMT protein contains 342 amino acids. The full-length PMT may be cloned into a T7-based expression plasmid (pET30a, Novagen) with a N-terminal His6 tag followed by a TEV protease cleavage site. This construct gives a low level of expression in the bacteria host BL21(DE3). Sequence analysis suggests a highly flexible region at the N-terminus of PMT. Successive removal of N-terminal amino acids from PMT lead to a construct with the N-terminal 19 residues truncated (named PMTΔ19 hereafter). This construct showed enhancement in the expression level. To further improve the expression and solubility, we co-expressed PMTΔ19 with GroEL in BL21(DE3). This strategy led to an improved yield of the soluble enzyme for induction with 0.5 mM IPTG either at room temperature or at 37° C. Truncation of the N-terminal 19 amino acids did not appear to have any effect on the enzyme activity as demonstrated by the in vitro enzymatic assay described earlier (FIG. 7).

The soluble fraction of the His6-tagged PMTΔ19 in the E. coli lysate may be purified first over a Ni-NTA agarose column and eluted using 250 mM imidazole. The N-terminal His6 tag may be removed by the TEV protease with a 1:20 molar ratio of protease to protein. After overnight cleavage at 4° C. with simultaneous dialysis against cell lysate buffer to remove imidazole, the uncleaved protein may be removed by Ni-NTA agarose column. The cleaved protein may fractionated on anion-exchange chromatography (Source-15Q, Pharmacia). PMTΔ19 does not bind to the Source-15Q column; thus the flow through from anion-exchange chromatography was concentrated by an ultrafiltration device and purified by gel filtration chromatography (Superdex-200, Pharmacia). The untagged protein may be purified to greater than 98% homogencity. The concentration of the protein may determined by spectroscopic measurement at 280 nm. A final yield of approximately 5-6 mg of pure PMT protein per liter of bacterial culture may be expected.

Crystallization and Data Collection of PMTΔ19

Diffracting crystals of PMTΔ19 may be grown at 4° C. by the banding-drop vapor-diffusion method by mixing PMTΔ19 (10 mg/mi) with an equal volume of reservoir solution containing 17-19% PEG2000 monomethyl ether (v/v), 150 mM triethylamine N-oxide, and 5 mM DTT. One tenth of the drop volume of 0.5 M potassium bromide was used as an additive to facilitate the growth of single crystals. The crystals appeared after 2 days and reached a maximum size within two weeks. The crystals are in the primitive space group P1, with unit cell parameters a=81.1 Angstroms, b=82.8 Angstroms, c=106.0 Angstroms, alpha=93.9°, beta=104.3°, gamma=105.4° and appear to contain 8-10 molecules in each asymmetric unit. Crystals of PMTΔ19 in the presence of 5 mM SAH, the enzymatic reaction product from SAM, are also obtained under similar conditions. The crystals are also in the primitive space group P1, but with unit cell parameters a=52.7 Angstroms, b=81.5 Angstroms, c=83.9 Angstroms, alpha=107.7°, beta 93.3°, gamma=103.5° and appear to contain 4 or 5 molecules in each asymmetric unit.

Diffraction data were collected using an ADSC quantum 315 CCD detector at the NSLS-X25 beamline at BNL. The crystals, appearing as irregular rods, diffract X-rays beyond 2.0 Angstrom resolution. To prevent potential crystal decay problem, we equilibrated the crystals in a cryoprotectant buffer containing 25% PEG2000 monomethyl ether (V/V), 150 mM triethylamine N-oxide, 5 mM DTT, and 15% glycerol, and flash-froze the crystals in liquid nitrogen. The crystals were kept frozen under nitrogen stream at −170° C. during data collection. A complete 2.0 Angstrom native data set was collected (Table 2).

TABLE 2
Crystallographic Analysis of PMTΔ19

| Data Sets | PMTΔ19 | PMTΔ19 + SAH |
|---|---|---|
| Wave length (Angstrom) | 1.10 | 1.10 |
| Resolution (Angstrom) | 99-2.0 | 99-2.2 |
| Unique reflections | 70,964 | 62,028 |
| Completeness (outer shell) | 97.5% (96.0%) | 95.4% (80.0%) |
| R_{sym} (outer shell) | 0.056 (0.3.62) | 0.058 (0.292) |
| Data redundancy | 3.8 | 1.9 |
| Average I/(sigma) (outer shell) | 26.4 (4.8) | 18.5 (3.0) |

### Example 3

### Biochemical Characterization of PME and the Regulation of PP2A Core Enzyme by PMT, PME, and Alpha4

Structural studies can reveal more functional insights when combined with biochemical characterization. We carried out biochemical characterization of the mechanisms and function of PME and how PP2A is regulated by PMT, PME, and the alpha4 protein.

Purification of Recombinant Proteins for Biochemical Characterization

We have purified recombinant PME, PMT, alpha4, and the PP2A core component, the A-C hetero-dimer from bovine brain (FIG. 8). Clones of PP2A A and C subunits and the alpha4 protein in different vectors/baculoviruses for expression in bacteria and insect cells are summarized in Table 3. Expression and solubility tests for these clones are also summarized in Table 3. Co-expression of the PP2A C subunit with the A subunit or the alpha4 protein using different expression vectors in bacteria or insect cells are summarized in Table 4. The A and C subunits of PP2A employed in this study are of the α isoform, which is more abundant than and shares very high sequence identity with the β isoform.

TABLE 3
Constructs of PP2A-A, PP2A-C, Alpha4 protein and their expression tests

| | | PP2A-A | | PP2A-C | | Alpha4 protein | |
|---|---|---|---|---|---|---|---|
| | | Expression | | Expression | | Expression | |
| Vectors/tag | Host | Level | Solubility | Level | Solubility | Level | Solubility |
| pGEX-2T/ | BL21 | +++ | +++ | +++ | − | ++ | ++ |
| N-GST | (DE3) | | | | | | |
| pET15b/ | BL21 | +++ | +++ | +++ | − | | |
| N-His6 | (DE3) | | | | | | |
| pET21b/ | BL21 | | | | | ++ | ++ |
| C-His6 | (DE3) | | | | | | |
| pET21b/ | BL21 | +++ | +++ | | | | |
| No tag | (DE3) | | | | | | |
| pET29b/ | BL21 | | | | | ++ | ++ |
| C-His6 | (DE3) | | | | | | |
| pACYCduet/ | BL21 | | | +++ | | | |
| N-His6 | (DE3) | | | | | | |
| pAcGHLT-B/ | insect | | | TBA | TBA | | |
| N-GST-his6 | cells | | | | | | |
| pVL1393/ | insect | +++ | +++ | | | ++ | ++ |
| no tag | cells | | | | | | |

TABLE 4
Co-expression of the C subunit of PP2A with the A subunit of PP2A or Alpha4 protein.

| Expression | Co-expression of PP2A-Cα with Aα or | | Expression level | |
|---|---|---|---|---|
| Host | Alpha4 expression vectors | | of PP2A-Cα | Solubility of PP2A-C |
| BL2I(DE3) | PP2A-A (pGEX-2T) | PP2A-C (in pACYCduet) | ++ (FIG. 11) | ++ (FIG. 11) |
| BL21(DE3) | Alpha4 (pGEX-2T) | PP2A-C (in pACYCduet) | ++ (FIG. 12) | ++ (FIG. 12) |
| BL21(DE3) | PP2A-A (pET21b) | PP2A-C (in pACYCduet) | ++ | ++ |
| insect ceils | PP2A-A (pVL1393) | PP2A-C (in pAcGHLT-B) | TBA | TBA |
| insect cells | Alpha4 (pVL1393) | PP2A-C (in pAcGHLT-B) | TBA | TBA |

Expression of Soluble A-C Hetero-Dimer in E. coli

We cloned both the A and C subunits into bacterial expression vectors. Using standard recombinant DNA technology, the full-length A subunit may be cloned into vectors pGEX-2T (Pharmacia), pET 15b (Novagen), and pET21b (Novagen), which share the pBR322 DNA replication origin. This enables us to express the A subunit with an N-terminal GST tag, and N-terminal His6 tag, and a C-terminal His6 tag, respectively (Table 3). The C subunit may also be cloned into the vectors listed above, as well as into pACYCduet (Novagen) with an N-terminal His6 tag (Table 3). The vector pACYCduet has a p15A DNA replication origins, and may be suitable for co-expression with the other three vectors listed above.

Expression of the A subunit is robust in the bacterial host BL21(DE3) for all clones and was purified to homogencity in large quantity using low temperature induction combined with co-expression with the A subunit. We may co-express pET15b-PP2A-A and pACYCduet-PP2A-C and purify A-C hetero-dimer protein on Ni-NTA resin as described herein.

Over-Expression and Purification of the Alpha4 Protein

The full-length alpha4 protein contains 339 amino acids. Although it binds to the C subunit of PP2A, alpha4 does not appear to contain any recognizable protein-protein interaction motif on the basis of sequence analysis. Using standard recombinant DNA technology, we cloned the coding DNA sequences of the alpha4 protein into three bacterial expression vectors pGEX-2T, pET21b, and pET29b, which may allow the expression of the alpha4 protein. C-terminal His6-tagged protein from the bacteria cell lysate may be purified over a Ni-NTA agarose column, followed by anion exchange chromatography (Source-15Q, Pharmacia) and gel filtration chromatography (Superdex-200, Pharmacia). After gel filtration, the protein may be further purified on a second anion exchange chromatography to greater than 98% homogencity. The concentration was determined by spectroscopic measurement at 280 nm. A final yield of approximately 5 mg of pure alpha-4 protein per liter of bacterial culture may be expected.

Expression and Purification of the Alpha4 Protein in Complex with the PP2A C Subunit

Co-expression of the C subunit of PP2A and the alpha4 protein may be performed by co-transformation of the bacteria host BL21(DE3) with a pACYCduet vector that produces the C subunit with a N-terminal His6 tag and a pGEX-2 T vector that expresses GST-tagged alpha4 protein or a pET21b vector that produces a C-terminal His6-tagged alpha4 protein. Induction of protein expression at a lower temperature (15-22° C.), but not at 37° C., gave an increased soluble fraction of the C subunit. This observation suggested that, similar to the A subunit, the alpha4 protein was capable of facilitating the folding of the C subunit in bacteria through mutual interactions. The soluble complex between the C subunit of PP2A and the alpha4 protein from the bacteria cell lysate was purified over Ni-NTA agarose column.

### Example 4

### Synthesis of Leucinal and LPMM

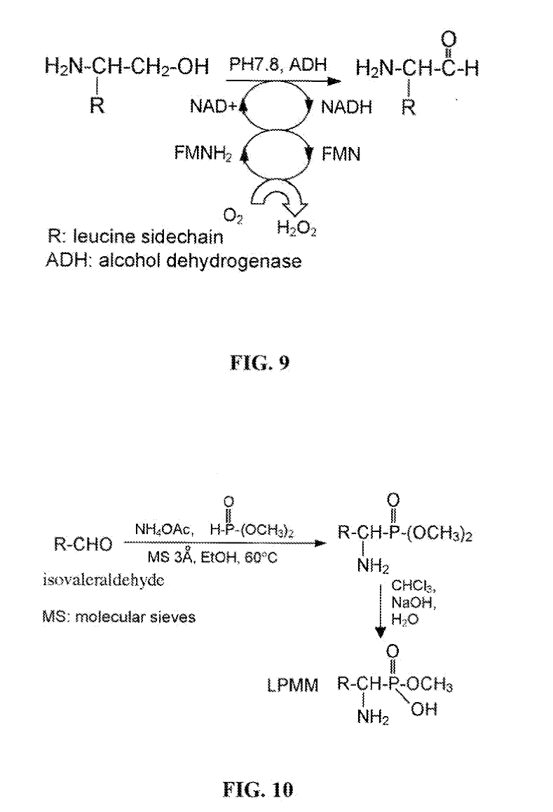

Synthesis of Leucinal follows an enzymatic reaction catalyzed by horse liver alcohol dehydrogenase (Andersson L et al. (1982) supra; Andersson L & Wolfenden R (1982) supra). Incubation of this enzyme with L-25 Leucinol in the presence of NAD+ and FMN may result in the oxidation of L-Leucinol and conversion to L-Leucinal (FIG. 9). Synthesis of LPMM involves treatment of isovaleraldehyde with an equal molar amount of ammonium acetate and dimethyl phosphite (Takahashi H et al. (1994) Synthesis 763-764) followed by hydrolysis at alkaline pH to remove one of the methyl group (Szewczyk J. (1982) Communications 409-412). The overall synthesis scheme is illustrated in FIG. 10. Briefly, the methanol solution of ammonium 30 acetate may be incubated with molecular sieves (3 Angstrom), isovaleraldehyde, and dimethyl phosphite at room temperature. The reaction mixture may be stirred at 60° C. for 44 hours, cooled to room temperature, and acidified to pH 1 with concentrated HCl. The solution may be washed with diethyl ether to remove neutral materials. The aqueous phase may be further stirred in the presence of CHCl₃ and 1N NaOH. The aqueous phase is LPMM and the organic phase contains the dimethyl compounds (FIG. 10).

Successful synthesis of the compounds may be verified by H¹-NMR, mass spectrometry and IR spectroscopy. These compounds may then be used as the carboxy-terminal immobilized residue instead of the Leucine reside to synthesize peptides with the sequence of the carboxyl terminal eight residues “RRTPDYFL”. The last six residues are the most conserved motif throughout all species. Standard procedure may be followed for the synthesis of the peptide derivatives.

### Example 5

### Generation of Missense Mutations of PME at Ser156

All three mutants of PME at position Ser156 may be cloned into a T7-based expression plasmid (pET 15b, Novagen) with an N-terminal His6 tag followed by a thrombin cleavage site. All mutant proteins may be expressed to high levels in the bacteria host (BL21DE3) after overnight induction at room temperature with 0.5 mM IPTG. The proteins may then be purified to homogencity by a Ni-NTA agarose column followed by anion-exchange chromatography (Source-15Q, Pharmacia). The concentrations of the proteins may be determined by spectroscopic measurement at 280 nm and the activity of the mutants measured using the assay described above.

While this disclosure has been disclosed with reference to specific embodiments, it is apparent that other embodiments and variations of this disclosure can be devised by other skilled in the art without departing from the true spirit and scope of the disclosure. The appended claims include all such embodiments and equivalent variations.

What is claimed is:

1. A compound comprising a mimetic of a peptide capable of binding to a binding pocket of a protein phosphatase 2A methyl esterase (PME), wherein the mimetic has a three dimensional structure complementary to the binding pocket of the PME defined by atomic coordinates of a substrate-PME complex.

2. The compound of claim 1, wherein the mimetic has a three dimensional structure which is represented by a model that deviates from the atomic coordinates of the c-terminus of the substrate by a root mean square deviation of less than 10 angstroms.

3. The compound of claim 2, wherein the c-terminus of the substrate is represented by a peptide which comprises amino acids involved in hydrogen bonding and van der Waals interactions with the residues in the binding pocket of PME.

4. The compound of claim 1, wherein a residue in the binding pocket of PME comprise Ser156.

5. The compound of claim 2, wherein the c-terminus of the substrate is represented by a computationally modeled peptide which comprises amino acids from the c-terminus of protein phosphatase 2A (PP2A).

6. The compound of claim 1, wherein the substrate is PP2A or a carboxyl-terminal portion of PP2A.

7. The compound of claim 1, wherein the mimetic is a peptide.

8. The compound of claim 1, wherein at least one amino acid of the peptide is replaced with a modified amino acid.

9. The compound of claim 1, wherein at least one bond in the peptide is replaced with a peptide bond substitute.

10. A peptide capable of binding to a binding pocket of a protein phosphatase 2A methyl esterase (PME).

11. The peptide of claim 10, wherein the binding pocket is a catalytic site.

12. The peptide of claim 10, wherein the peptide is Arg-Arg-Thr-Pro-Asp-Tyr-Phe-Z, where Z may be Leucine, Leucinal or LPMM.

13. The peptide of claim 10, wherein the peptide is capable of inhibiting PME catalytic activity.

14. A synthetic peptide comprising the sequence selected from the group consisting of

| | |
|---|---|
| X₁X₂X₃X₄X₅X₆X₇X₈; | (SEQ ID No. 8) |
| X₂X₃X₄X₅X₆X₇X₈; | (SEQ ID No. 9) |
| X₃X₄X₅X₆X₇X₈; | (SEQ ID No. 10) |
| X₄X₅X₆X₇X₈; | (SEQ ID No. 11) |
| and | |
| X₅X₆X₇X₈; | (SEQ ID No. 12) |

wherein X₁ is Arg, Lys or Gln or a mimetic of Arg, Lys or Gln; X₂ is Arg, Lys or Gln or a mimetic of Arg, Lys or Gln; X₃ is Thr, Ser or Val or a mimetic of Thr, Ser or Val; X₄ is Pro or Ala or a mimetic of Pro or Ala; X₅ is Asp or Glu or a mimetic of Asp or Glu; X₆ is Tyr or Phe or a mimetic of Tyr or Phe; X₇ is Phe or Tyr or a mimetic of Phe or Tyr; and X₈ is Leu, Ile, Val, Leucinal or LPMM or a mimetic of Leu, Ile, Val, Leucinal or LPMM.

15. The synthetic peptide of claim 14, wherein the sequence X₁X₂X₃X₄X₅X₆X₇X₈ is Arg-Arg-Thr-Pro-Asp-Tyr-Phe-Leu (SEQ ID No. 3).

16. The synthetic peptide of claim 14, wherein the sequence X₃X₄X₅X₆X₇X₈ is Thr-Pro-Asp-Tyr-Phe-Leu (SEQ ID No. 7).

17. The synthetic peptide of claim 14, wherein the sequence X₅X₆X₇X₈ is Asp-Tyr-Phe-Leu (SEQ ID No. 6).

18. The synthetic peptide of claim 14, wherein at least one bond in the peptide is replaced with a peptide bond substitute.

19. The synthetic peptide of claim 14, wherein the peptide inhibits catalytic activity of PME.

20. A method of identifying a compound that inhibits catalytic activity of protein phosphatase 2A methylesterase (PME), comprising:
obtaining a set of atomic coordinates defining a three dimensional structure of a crystal of a substrate-PME complex that effectively diffracts X-rays for the determination of atomic coordinates to a resolution of 5 Angstroms or better:
selecting a compound that mimics the substrate binding to the catalytic site on the PME by performing structure based drug design with the atomic coordinates obtained in step (a), wherein said selecting is performed in conjunction with computer modeling;
contacting the compound with the PME; and
detecting binding of the compound with the catalytic site of the PME, wherein the compound is selected if it is capable of inhibiting PME catalytic activity.

21. The method of claim 20, wherein selecting performed in conjunction with computer modeling is selecting a mimetic which is represented by a model that deviates from the atomic coordinates of the substrate by a root mean square deviation of less than 10 angstroms, wherein the substrate is represented by a peptide which comprises amino acids involved in hydrogen bonding and van der Waals interactions with the catalytic site of PME.

22. The method of claim 20, wherein performing structure based drug design comprises computational screening of one or more databases of chemical compound structure to identify candidate compounds which have structures that are predicted to interact with the catalytic site of the PME.

23. The method of claim 20, wherein the substrate is PP2A or a carboxyl-terminal portion of PP2A.


SEQUENCE LISTING (12 sequences)

SEQ ID NO: 1 - protein, 8 aa, Artificial Sequence
  Carboxy-terminal eight residues of the PP2A C subunit from human
  misc_feature (8)..(8): Xaa is Leucinal
RRTPDYFX

SEQ ID NO: 2 - protein, 8 aa, mammalian
  Carboxy-terminal eight residues of the PP2A C subunit
RRTPDYFL

SEQ ID NO: 3 - protein, 8 aa, Artificial Sequence
  LPMM is phosphonic acid analogue of leucine monomethyl ester
  misc_feature (8)..(8): Xaa is phosphonic acid analogue of leucine monomethyl ester
RRTPDYFX

SEQ ID NO: 4 - protein, 9 aa, mammalian
  Carboxy terminal nine residues from PP2A C subunit
TRRTPDYFL

SEQ ID NO: 5 - protein, 10 aa, mammalian
  Carboxy terminal ten residues from PP2A C subunit
VTRRTPDYFL

SEQ ID NO: 6 - protein, 4 aa, mammalian
  Carboxy terminal four residues from PP2A C subunit
DYFL

SEQ ID NO: 7 - protein, 6 aa, mammalian
  Carboxy terminal six residues from PP2A C subunit
TPDYFL

SEQ ID NO: 8 - protein, 8 aa, Artificial Sequence
  Carboxy-terminal eight residues of the PP2A C subunit from human
  misc_feature (1)..(2): Xaa is Arg, Lys or Gln or a mimetic of Arg, Lys or Gln
  misc_feature (3)..(3): Xaa is Thr, Ser or Val or a mimetic of Thr, Ser or Val
  misc_feature (4)..(4): Xaa is Pro or Ala or a mimetic of Pro or Ala
  misc_feature (5)..(5): Xaa is Asp or Glu or a mimetic of Asp or Glu
  misc_feature (6)..(6): Xaa is Tyr or Phe or a mimetic of Tyr or Phe
  misc_feature (7)..(7): Xaa is Phe or Tyr or a mimetic of Phe or Tyr
  misc_feature (8)..(8): Xaa is Leu, Ile, Val Leucinal or phosphonic acid analogue of leucine monomethyl ester or a mimetic of Leu, Ile, Val, Leucinal or phosphonic acid analogue of leucine monomethyl ester
XXXXXXXX

SEQ ID NO: 9 - protein, 7 aa, Artificial Sequence
  Carboxy-terminal seven residues of the PP2A C subunit from human
  misc_feature (1)..(1): Xaa is Arg, Lys or Gln or a mimetic of Arg, Lys or Gln
  misc_feature (2)..(2): Xaa is Thr, Ser or Val or a mimetic of Thr, Ser or Val
  misc_feature (3)..(3): Xaa is Pro or Ala or a mimetic of Pro or Ala
  misc_feature (4)..(4): Xaa is Asp or Glu or a mimetic of Asp or Glu
  misc_feature (5)..(5): Xaa is Tyr or Phe or a mimetic of Tyr or Phe
  misc_feature (6)..(6): Xaa is Phe or Tyr or a mimetic of Phe or Tyr
  misc_feature (7)..(7): Xaa is Leu, Ile, Val, Leucinal or phosphonic acid analogue or a mimetic of Leu, Ile, Val, Leucinal or phosphonic acid analogue of leucine monomethyl ester
XXXXXXX

SEQ ID NO: 10 - protein, 6 aa, Artificial Sequence
  Carboxy-terminal six residues of the PP2A C subunit from human
  misc_feature (1)..(1): Xaa is Thr, Ser or Val or a mimetic of Thr, Ser or Val
  misc_feature (2)..(2): Xaa is Pro or Ala or a mimetic of Pro or Ala
  misc_feature (3)..(3): Xaa is Asp or Glu or a mimetic of Asp or Glu
  misc_feature (4)..(4): Xaa is Tyr or Phe or a mimetic of Tyr or Phe
  misc_feature (5)..(5): Xaa is Phe or Tyr or a mimetic of Phe or Tyr
  misc_feature (6)..(6): Xaa is Leu, Ile, Val Leucinal or phosphonic acid analogue of leucine monomethyl ester or a mimetic of Leu, Ile, Val, Leucinal or phosphonic acid analogue of leucine monomethyl ester
XXXXXX

SEQ ID NO: 11 - protein, 5 aa, Artificial Sequence
  carboxy-terminal five residues of the PP2A C subunit from human
  misc_feature (1)..(1): Xaa is Pro or Ala or a mimetic of Pro or Ala
  misc_feature (2)..(2): Xaa is Asp or Glu or a mimetic of Asp or Glu
  misc_feature (3)..(3): Xaa is Tyr or Phe or a mimetic of Tyr or Phe
  misc_feature (4)..(4): Xaa is Phe or Tyr or a mimetic of Phe or Tyr
  misc_feature (5)..(5): Xaa is Leu, Ile, Val Leucinal or phosphonic acid analogue of leucine monomethyl ester or a mimetic of Leu, Ile, Val, Leucinal or phosphonic acid analogue of leucine monomethyl ester
XXXXX

SEQ ID NO: 12 - protein, 4 aa, Artificial Sequence
  carboxy-terminal four residues of the PP2A C subunit from human
  misc_feature (1)..(1): Xaa is Asp or Glu or a mimetic of Asp or Glu
  misc_feature (2)..(2): Xaa is Tyr or Phe or a mimetic of Tyr or Phe
  misc_feature (3)..(3): Xaa is Phe or Tyr or a mimetic of Phe or Tyr
  misc_feature (4)..(4): Xaa is Leu, Ile, Val Leucinal or phosphonic acid analogue of leucine monomethyl ester or a mimetic of Leu, Ile, Val, Leucinal or phosphonic acid analogue of leucine monomethyl ester
XXXX